(19) United States
(12) Patent Application Publication
(10) Pub. No.: US 2010/0217091 A1
(43) Pub. Date: Aug. 26, 2010

(54) Vaginal speculum providing an unobstructed view

(75) Inventor: Stephen J. Sullivan, Redmond, WA (US)

Correspondence Address: BLACK LOWE & GRAHAM, PLLC, 701 FIFTH AVENUE, SUITE 4800, SEATTLE, WA, 98104, US

(73) Assignee: Gynex Corporation

(21) Appl. No.: 12/378,865

(22) Filed: Feb. 20, 2009

(51) Int. Cl. A61B 1/32

(52) U.S. Cl. 600/222

(57) ABSTRACT

The invention relates to a vaginal speculum including an upper assembly and a lower assembly. The upper assembly includes a first spoon portion and a single support bar connecting the first spoon portion to the first handle portion. The lower assembly includes a second spoon portion and a second handle portion. The single support bar of the upper assembly extends distally and in a posterior direction from the first and second spoon portions to provide an unobstructed view of an area between the first spoon portion and the second spoon portion.

### FIELD OF THE INVENTION

This invention generally relates to a vaginal speculum for use during surgical and examination procedures. A vaginal speculum includes two parallel spoon portions that can be inserted into a woman's vagina and then spread apart by operation of a speculum handle mechanism. The spread speculum allows for a physician to view and operate on areas within the vagina that would otherwise be inaccessible.

### BACKGROUND OF THE INVENTION

The vaginal speculum is the most commonly used device in the field of gynecology today. It is routinely utilized for observation during a vaginal examination or for accessibility during various gynecological surgeries and treatments. Typically, the spoon portions of the speculum are lubricated and then inserted into a woman's vagina in the closed position. After insertion, the handle portions of the speculum can then be operated with a compression action to spread and lock the inserted spoon portions apart within the vagina. This action increases the diameter of the vagina, allowing a physician to observe and access an area of interest within the vagina or on the cervix through an enclosed aperture within the speculum. The aperture in the spread speculum is surrounded by the upper and lower spoon portions in combination with two connecting support elements on either side of the speculum, which connect the upper spoon portion to the lower spoon portion and a handle portion.

Unfortunately, vaginal specula today have an opening that confines a physician during examination and medical procedures. Even those specula having only a single side support are structured such that the side support is in immediate proximity to the spoon portions which confines the physician. For example, during a surgery of a cervix, where the simultaneous use of multiple surgical instruments may be required, the aperture between the speculum spoon and the side support element portions only permits a small angle of instrument insertion. In this configuration, the inserted medical instruments in the previous specula openings can substantially block a physician's view of and access to an area of interest.

Therefore, it would be advantageous to have an improved vaginal speculum that provides a substantially unhindered view of an area within a vagina under observation during a routine medical procedure. Further, it would be helpful for the improved speculum to incorporate a practical single-hand method of manipulation of medical instruments used during the procedure, while still providing an unobstructed view of the area under observation.

### SUMMARY OF THE INVENTION

The invention relates to a vaginal speculum including an upper assembly and a lower assembly. In accordance with one aspect of the invention, the upper assembly includes a first spoon portion and a single support bar connecting the first spoon portion to the first handle portion. The lower assembly includes a second spoon portion and a second handle portion. The single support bar of the upper assembly provides an unobstructed view of an area between the first spoon portion and the second spoon portion.

In accordance with another aspect of the invention, the single support bar is the only connecting support element of the upper assembly.

In accordance with a further aspect of the invention, the single support bar is positioned to only one side of the first spoon portion and the first handle portion.

In accordance with another aspect of the invention, the support bar is located to a side of and curved at a posterior angle to the direction of the first and second spoon portions.

In accordance with yet another aspect of the invention, the first handle portion includes a spring-loaded ratchet locking mechanism that locks the upper assembly to the lower assembly.

In accordance with a further aspect of the invention, the ratchet locking mechanism is single-hand operable.

In accordance with yet another aspect of the invention, the first handle portion encloses a portion of the second handle portion, such that the first handle portion can be adjusted by sliding the first handle portion over a portion of the second handle portion while remaining secured to the second handle portion.

### BRIEF DESCRIPTION OF THE DRAWINGS

Preferred and alternative examples of the present invention are described in detail below with reference to the following drawings:

FIG. 1 illustrates a perspective view of a vaginal speculum formed in accordance with an embodiment of the present invention;

FIG. 2 illustrates a side view of the vaginal speculum;

FIG. 3 illustrates a cross-sectional side view of a ratchet locking mechanism of the vaginal speculum; and

FIG. 4 illustrates a top view of the vaginal speculum.

### DETAILED DESCRIPTION

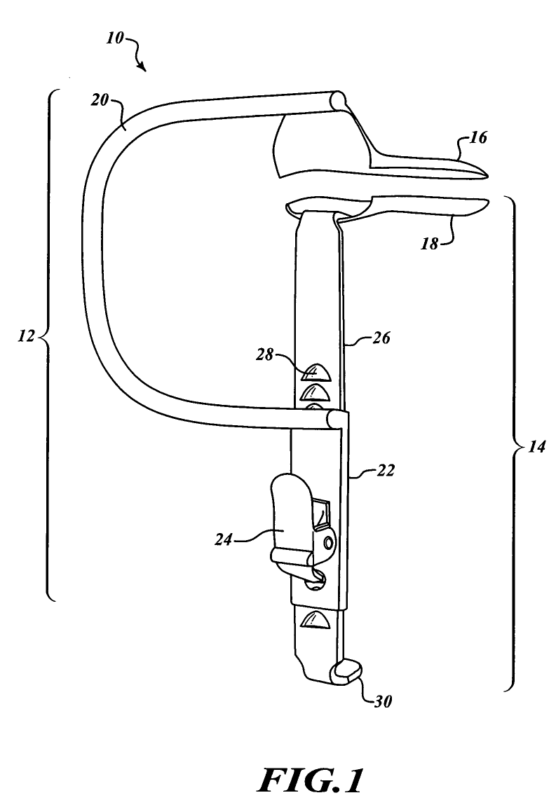

FIG. 1 illustrates a vaginal speculum 10 formed in accordance with an example embodiment of the present invention. The vaginal speculum 10 includes an upper assembly 12 that is slidably connected with a lower assembly 14. The upper assembly 12 includes an upper spoon portion 16, a single support bar 20, and an upper handle portion 22 with a locking mechanism 24. The single support bar 20 as shown is an angled c-arm, but may have a different shape in other embodiments. The upper assembly 12 is structured as a single unit such that the upper spoon portion 16, the single support bar 20, and the upper handle portion 22 are in a fixed, non-adjustable relationship to each other. The single support bar 20 is formed of a curved rigid rod in the example embodiment shown, but may have different shapes in other embodiments. The lower assembly 14 includes a lower spoon portion 18 and a lower handle portion 26 with a plurality of notches 28 and a handle seat 30. The upper spoon portion 16 and the lower spoon portion 18 may also be referred to as upper and lower blades, respectively. Although the upper spoon portion 16 and the lower spoon portion 18 are shown to have a particular shape, they may also have other shapes and/or sizes in other embodiments. The single support bar 20 connects the upper spoon portion 16 to the upper handle portion 22 and is curved such that it extends in a distal and posterior direction from the upper and lower spoon portions 16, 18. One or more tubes (not shown) may be positioned on at least one of the upper and lower spoon portions 16, 18 for drawing fluids and/or smoke from the vaginal area during procedures. An illumination source (not shown) that may include a fiber optic bundle (not shown) along at least one of the upper or lower spoon portion 16, 18 may also be included. The vaginal speculum 10 is preferably made of stainless steel, but may also be made of other materials such as polymer plastics or composite materials and/or include an insulating layer for electrosurgical or laser procedures.

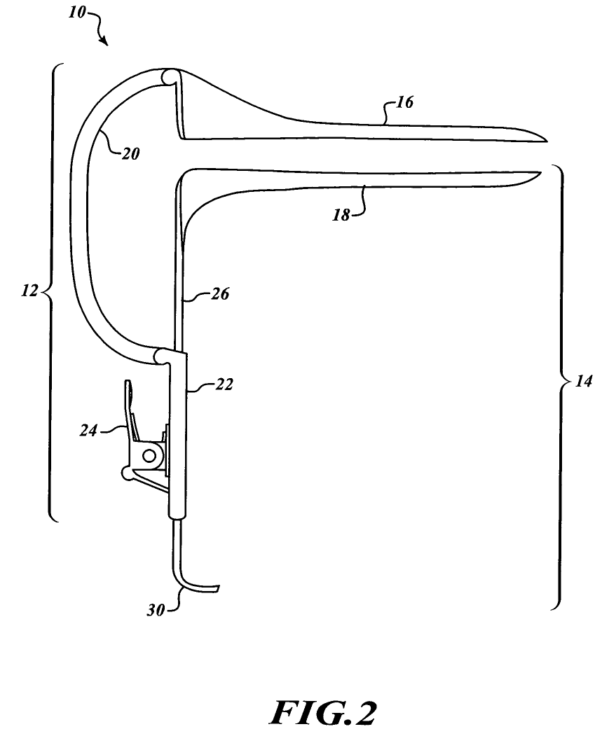

FIG. 2 illustrates that the upper handle portion 22 of the upper assembly 12 covers a region of the lower handle portion 26 of the lower assembly 14 in order to facilitate a secure sliding adjustment affecting the spacing between the upper spoon portion 16 and the lower spoon portion 18. The upper handle portion 22 is grooved along its interior sides in a manner that allows the sides of the lower handle portion 26 to fit within the grooves to provide a wrapped-fit. This wrapped-fit allows the grooved upper handle portion 22 to slide along the length of lower handle portion 26 in upward and downward translational directions. In one embodiment, the upper handle portion 22 is configured to completely enclose a region of the lower handle portion 26. However, in other embodiments, the upper handle portion 22 may be configured to only partially enclose a region of the lower handle portion 26.

The upper assembly 12 may be removed from the lower assembly 14 by moving the upper assembly 12 in the upward translational direction such that the upper handle portion 22 slides off of the lower handle portion 24. However, in other embodiments, the vaginal speculum 10 may be structured to prevent removal of the upper assembly 12. The handle seat 30 stops movement of the upper assembly 12 in the downward translational direction. The handle seat 30 is in the form of a bend at an end of the lower handle portion 26 farthest from the lower spoon portion 18. At a position where the upper handle portion 22 comes into contact with the handle seat 30, the upper spoon portion 16 is in a closed position with the lower spoon portion 18. However, in other embodiments, the handle seat 30 may be formed in a different manner or may be absent, with the contact of the upper spoon portion 16 with the lower spoon portion 18 stopping movement of the upper assembly 12 in the downward translational direction. After being moved to a desired position, the upper assembly 12 can be locked to the lower assembly 14 with the locking mechanism 24 of the upper handle portion 22.

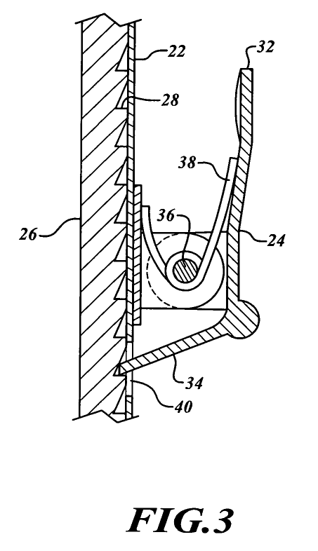

FIG. 3 shows additional detail for the locking mechanism 24. The locking mechanism 24 includes a release handle 32 and a tooth 34. The locking mechanism 24 is illustrated in a normal locked position, with the tooth 34 of the locking mechanism 24 fixed inside one of the plurality of notches 28 of the lower handle portion 26, through a hole 40 in the upper handle portion 22. The notches 28 are angled ratchet notches that allow movement of the upper handle portion 22 in only an upward translational direction when the locking mechanism 24 is in the locked position. Applying an adequate upward force to the tooth 34 (having a raised thumb grip) causes the ratchet locking mechanism 24 to slide upward over the angled notches 28, thereby opening the speculum spoon portions 16 and 18. The tooth 34 slides across the angled notches 28, until pressure is no longer applied. At this point, the tooth 34 catches in the angled notch 28 aligned with the hole 40 in the upper handle portion 22 to hold the speculum 10 open with a preferred distance between the spoon portions 16 and 18. A spring 38 wrapped around a pivot axle 36 of the locking mechanism 24 applies a mechanical force to move the tooth 34 against the lower handle portion 26. Subsequent pressure from the vagina forces the tooth 34 into one of the notches 28 thereby locking the speculum 10 in the desired position.

When a user depresses the release handle 32, the tooth 34 is lifted away from the lower handle portion 26 and therefore is released from the angled locking notch 28, allowing the upper handle portion 22 to slide in the downward translational direction over the lower handle portion 26. This downward movement causes the speculum spoon portions 16 and 18 to draw closer together, such that the speculum 10 can be closed and removed from the vagina after an examination or a medical procedure. Releasing the downward force applied to the release handle 32 at any point during a sliding adjustment, causes the tooth 34 to fall into one of the plurality of angled locking notches 28 on the lower handle portion 26 and the speculum 10 to thereby remain open at the desired distance.

The notches 28 on the lower handle portion 26 are spaced a predetermined distance apart such that a desired level of precision is achieved relating to the locked separation between the upper and lower spoon portions 16 and 18. The notches 28 on the lower handle portion 26 are spaced approximately 5 millimeters (mm) apart from the next adjacent notch on the handle 26. Such precision adjustment of the speculum spoon portions 16 and 18 allows for a variety of open positions to be achieved to better accommodate specific patients and procedural requirements. For example a routine vaginal examination may not require the same degree of dilation as would a cervical surgery, where maximal dilation may be required.

While the locking mechanism 24 is illustratively depicted in FIGS. 1-3, the locking mechanism of the present invention is not limited to this particular embodiment. In accordance with other embodiments of the invention, the locking mechanism can include other single-hand operable spring or lever activated locking mechanisms, a different spacing between the notches 28, and/or other types of locking mechanisms without departing from the spirit and scope of the present invention (e.g., a ball and spring locking mechanism).

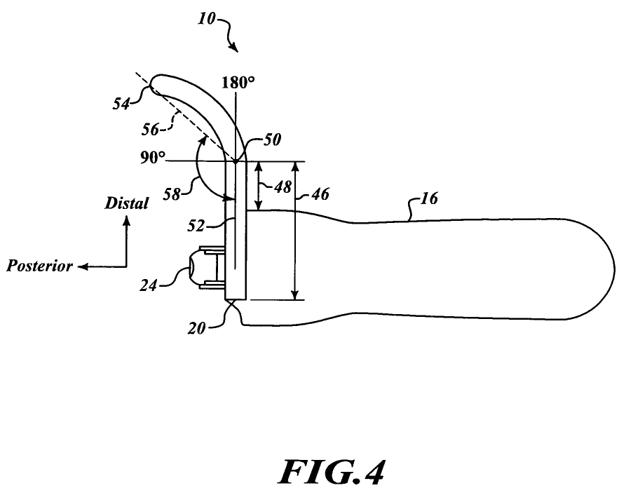

FIG. 4 illustrates a top view of the vaginal speculum 10. A directional indicator shows that a posterior direction is defined as extending behind the first spoon portion 16 and the second spoon portion 18 (not shown in this figure). The directional indicator also shows that a distal direction is defined as being perpendicular to the posterior direction, in a lateral direction away from the first spoon portion 16 and the second portion 18 (not shown in this figure). Although the distal direction is shown extending to the left of the speculum 10 viewing the speculum 10 from behind, it should be understood that the distal direction would extend in the opposite direction, to the right of the speculum 10, in an embodiment where the support bar 20 extends to the right rather than the left of the first spoon portion 16 when viewing the speculum 10 from behind.

The vaginal speculum 10 has a curved bend in the single support bar 20, which can accommodate for patient anatomy while simultaneously providing an unhindered view and access to a region of a vagina under examination. The support bar 20 extends a first distance 46 from an end of the support bar and a second distance 48 from an edge of the first spoon portion 16 before the bend begins. In the example embodiment shown, the first distance 46 is approximately 5.5 centimeters (cm), but the first distance 46 and/or the second distance 48 may vary or even be zero in some embodiments. For example, the second distance 48 may be zero with the bend starting at an edge of the upper spoon portion 16. A point 50 is shown to illustrate a location along the length of the single support bar 20 where the bend begins. A reference axis 52 extends through a central axis of the single support bar 20 before the support bar 20 begins to bend and passes through the point 50. A point 54 is shown to illustrate a most posterior point of the support bar 20 when viewed from above. A reference line segment 56 extends from the point 50 to the point 54. In various embodiments, the bend of the single support bar 20 can be configured at an angle 58 ranging between approximately 90° and approximately 145° as measured in a clockwise direction from the reference axis 52 to the reference line segment 56. However, other angles may be used. The angle 58 is preferably between 110° and 130° and is approximately 115° in an example embodiment. Additionally, it should be understood that the angle 58 would be measured in a counter-clockwise direction for an embodiment where the support bar 20 extends to the right rather than the left of the first spoon portion 16 when viewing the speculum 10 from behind.

The single support bar 20 is firmly attached (e.g., by weld or mold) to an upper region of the upper spoon portion 16 and to an upper region of the upper handle portion 22, to prevent the possibility of the upper assembly 12 bending or breaking under pressure. The single support bar 20 is configured to provide sufficient strength based on an appropriate shape and/or thickness to provide sufficient support to the upper assembly 12 in the presence of vaginal pressure. The single support bar 20 may be formed of a metal such as stainless steel or formed of a plastic material, for example. Because the single support bar 20 is located distally from the upper and lower spoon portions 16, 18 of the speculum 10 and is bent at a posterior angle, the single support bar 20 both allows an unobstructed view and does not interfere with patient anatomy, namely the leg of the patient, during use.

In FIGS. 1, 2 and 4, the single support bar 20 is illustrated as being positioned to the left side of the speculum. This configuration is preferred by most right-handed physicians. However, in an alternate embodiment, the single support bar is positioned on the right side of the speculum. This variation can accommodate a physician who has a preference for having the support bar on the right side of the speculum, as do most left-handed physicians.

While several embodiments of the present invention have been illustrated and described herein, many changes can be made without departing from the spirit and scope of the invention. For example, different curve structures or non-curved structures such as straight angled structures or structures having multiple angled segments when viewed from above may be used for the single support bar 20 so long as the single support bar 20 extends distally and in a posterior direction from the spoon portions 16, 18. Accordingly, the scope of the invention is not limited by any disclosed embodiment. Instead, the scope of the invention should be determined from the appended claims that follow.

The embodiments of the invention in which an exclusive property or privilege is claimed are defined as follows:

1. A vaginal speculum, comprising:
an upper assembly comprising:
a first spoon portion;
a first handle portion; and
a single support bar connecting the first spoon portion to the first handle portion; and
a lower assembly comprising:
a second spoon portion; and
a second handle portion slidably coupled to the first handle portion,
wherein the single support bar is positioned on only one side of the first spoon portion, wherein the support bar is in a fixed non-adjustable relationship with respect to the first spoon portion and the first handle portion, and wherein the single support bar extends distally and in a posterior direction from the first and second spoon portions,
whereby the single support bar provides an unobstructed view of an area between the first spoon portion and the second spoon portion.

2. The vaginal speculum of claim 1, wherein the single support bar is the only element connecting the first spoon portion to the first handle portion.

3. The vaginal speculum of claim 2, wherein the single support bar is an angled c-arm.

4. The vaginal speculum of claim 1, wherein the single support bar is attached to an upper portion of the first spoon portion and to an upper portion of the first handle portion.

5. The vaginal speculum of claim 1, wherein the single support bar is of curved shape.

6. The vaginal speculum of claim 1, wherein the support bar extends at an angle between approximately 90 degrees and approximately 145 degrees from a reference axis that passes through a central axis of the support bar before the support bar begins to bend when viewed from above the first spoon portion, wherein the angle is measured between the reference axis and a reference line segment that extends from the reference axis where the support bar begins to bend to a most posterior point of the support bar when viewed from above the first spoon portion.

7. The vaginal speculum of claim 6, wherein the angle is between approximately 110 and 130 degrees.

8. The vaginal speculum of claim 7, wherein the angle is approximately 115 degrees.

9. The vaginal speculum of claim 1, wherein the first handle portion further comprises a locking mechanism that locks the upper assembly to the lower assembly.

10. The vaginal speculum of claim 9, wherein the locking mechanism is a spring loaded ratchet locking mechanism.

11. The vaginal speculum of claim 10, wherein the loaded ratchet locking mechanism is single-hand operable.

12. The vaginal speculum of claim 9, wherein the second handle portion comprises notches wherein the locking mechanism can be secured.

13. The vaginal speculum of claim 1, wherein the first handle portion encloses a portion of the second handle portion, such that the first handle portion can be can be slidably adjusted over a portion of the second handle portion while remaining secured to the second handle portion.

14. A vaginal speculum, comprising:
an upper assembly comprising:
a first spoon portion;
a first handle portion; and
a support element consisting of a single support bar connecting the first spoon portion to the first handle portion; and
a lower assembly comprising:
a second spoon portion; and
a second handle portion slidably coupled to the first handle portion,
wherein the single support bar is positioned on only one side of the first spoon portion and wherein the single support bar extends distally and in a posterior direction from the first and second spoon portions.

15. The vaginal speculum of claim 14, wherein the support bar extends at an angle between approximately 90 degrees and approximately 145 degrees from a reference axis that passes through a central axis of the support bar before the support bar begins to bend when viewed from above the first spoon portion, wherein the angle is measured between the reference axis and a reference line segment that extends from the reference axis where the support bar begins to bend to a most posterior point of the support bar when viewed from above the first spoon portion.

16. The vaginal speculum of claim 15, wherein the angle is between approximately 110 and 130 degrees.

17. The vaginal speculum of claim 16, wherein the angle is approximately 115 degrees.

18. The vaginal speculum of claim 14, wherein the first handle portion further comprises a locking mechanism that locks the upper assembly to the lower assembly.

19. The vaginal speculum of claim 18, wherein the locking mechanism is a spring loaded ratchet locking mechanism.

20. The vaginal speculum of claim 18, wherein the second handle portion comprises notches wherein the locking mechanism can be secured.

21. A vaginal speculum comprising:
an upper assembly comprising:
a first spoon portion;
a first handle portion; and
not more than one support bar connecting the first spoon portion to the first handle portion; and
a lower assembly comprising:
a second spoon portion; and
a second handle portion slidably coupled to the first handle portion,
wherein the support bar is positioned on only one side of the first spoon portion, wherein the support bar is in a fixed, non-adjustable relationship with respect to the first spoon portion and the first handle portion, and wherein the support bar extends distally and in a posterior direction from the first and second spoon portions,
whereby the support bar provides an unobstructed view of an area between the first spoon portion and the second spoon portion.

22. A vaginal speculum comprising:
an upper assembly comprising:
a first spoon portion;
a first handle portion; and
not more than one support bar connecting the first spoon portion to the first handle portion; and
a lower assembly comprising:
a second spoon portion; and
a second handle portion slidably coupled to the first handle portion,
wherein the support bar is positioned on only one side of the first spoon portion, wherein the support bar is formed of a rigid curved rod, and wherein the support bar extends distally and in a posterior direction from the first and second spoon portions,
whereby the support bar provides an unobstructed view of an area between the first spoon portion and the second spoon portion.

23. A vaginal speculum, comprising:
an upper assembly comprising:
a first spoon portion;
a first handle portion;
a single curved support bar connecting the first spoon portion to the first handle portion; and
a spring loaded ratchet locking mechanism configured to be operable with a single hand, the locking mechanism attached to the first handle portion; and
a lower assembly comprising:
a second spoon portion; and
a second handle portion having notches configured to cooperate with the spring loaded ratchet locking mechanism such that the spring loaded ratchet locking mechanism locks the upper assembly with respect to the lower assembly,
wherein the single support bar is the only element connecting the first spoon portion to the first handle portion and is positioned on only one side of the first spoon portion, wherein the single support bar extends distally and in a posterior direction from the first and second spoon portions, and wherein the first handle portion encloses a portion of the second handle portion, such that the first handle portion can be slidably adjusted over a portion of the second handle portion while remaining secured to the second handle portion when the spring loaded ratchet locking mechanism is in a released position.
